(19) United States
(12) Patent Application Publication
(10) Pub. No.: US 2013/0087158 A1
(43) Pub. Date: Apr. 11, 2013

(54) Medical Restraint Device

(71) Applicant: Lindsey Clay Laughter, North Hollywood, CA (US)

(72) Inventor: Lindsey Clay Laughter, North Hollywood, CA (US)

(21) Appl. No.: 13/271,186

(22) Filed: Oct. 11, 2011

(51) Int. Cl. A61F 5/37

(52) U.S. Cl. 128/876

(57) ABSTRACT

A non-behavioral medical restraint which keeps the patient in proper alignment in the bed.

### FIELD OF THE INVENTION

The present invention relates to a non-behavioral medical restraint which keeps the patient in proper alignment in the bed.

### BACKGROUND OF THE INVENTION

U.S. Pat. No. 3,829,079 relates to a spinal position patient restraint. A patient lying on their side on a medical treatment table in a curled position with bowed spine, is prevented from involuntarily or inadvertently kicking, straightening the spine, or otherwise making a movement potentially dangerous to the spine. A cervical-occipital pad and a posterior thigh pad are provided to bear against general anatomical portions of the patient. Each pad is individually mounted for vertical, horizontal, and pivotal adjustment being brought to the correct position with respect to the patient. Selectively adjustable fixing or locking means are provided. Various mechanical expedients, including sliding, or telescoping, or threaded connections to carry out these functions are provided.

U.S. Pat. No. 4,045,678 relates to a medical restraint assembly for holding a portion of a patient in a fixed position on an x-ray table for taking x-rays. The assembly comprising a restraint and restraint support. The restraint includes a platform which has a plurality of suction cups for mounting the platform to the x-ray table, restraint support, or other planar surface. A padded surface overlies at least a portion of the platform and receives and supports the portion of the patient to be restrained. A pair of sidewalls are attached to the platform on opposite sides of the padded surface and can be moved together to support the portion of the patient to be restrained. If desired, a pair of slots is formed in the platform on the opposite sides of the padded surface and a strap pass through the slots and around the portion of the patient to be restrained. The restraint support includes a back plate that is removably mounted perpendicular to the x-ray table and a front plate slidably coupled parallel and adjacent to the back plate for vertical movement relative to the table.

U.S. Pat. No. 4,885,811 relates to a restraint comprising a multiplicity of flexible bubbles which are encased in an airtight envelope of film material. When air inside the envelope is withdrawn, the restraint molds to animate or inanimate object to which it surrounds.

U.S. Pat. No. 5,012,821 relates to a medical restraint apparatus for use in securing a patient to a bed. The apparatus including a web strap which extends across the width of the bed and secured at opposite ends of the bed frame. A pair of cuffs are mounted on the web strap at spaced locations such that the patient wrists will be held at its side.

U.S. Pat. Nos. 5,242,377 and 5,314,404 relate to a head and neck support device. The device includes a first plate and a second plate. The first plate extends along the back of the torso and is strapped to the torso by a torso harness. The second plate extends upward from the first plate behind the head and neck. A head harness for grasping the head is attached to the device by a tether which is inserted through slots in a right and left harness support mounted on the second plate. The head harness allows the head to rotate while maintaining the head in a desired position.

U.S. Pat. No. 5,248,293 relates to a support device for people who suffer from neck dysfunction resulting in loss of head control. The support device includes a stiff support means mounted on the torso such as a yoke worn over the shoulders and the upper torso, and a mast that extends upward from the yoke, behind the wearer's head to a rear portion of the head at a height equal to the head's center of gravity. A head harness is worn on the head. A tether system consists of one or more tethers attached between a slider line mounted on the head harness and a portion of the mast adjacent the rear portion of the head.

U.S. Pat. No. 5,329,934 relates to a medical patient restraint device for additional comfort while restraining patients during surgical procedures such as the circumcision of infants. The patient restraint device has a planar support member sized to receive and support a patient in a lying position. The support member defines opposite side edges. A pair of support straps are fixed to the support member and extend along side edge portions in a parallel relationship. The pair of support straps are laterally spaced apart a distance sufficient to receive the patient between the support straps. Individual restraining straps are provided for releasable connection to the patients arms and legs. Mating fasteners are fixed to the pair of support straps and the individual restraining straps such that each of the restraining straps may be releasably attached to one of the pair of support straps so as to permit the individual straps to retain the patient in a spread eagle position on the support member without having the individual restraining straps extending toward the torso of the patient.

U.S. Patent Publication 20070012318 relates to a medical restraint device used to secure the arms, hands and wrists of patients that have lost control of their upper extremities. The device positions the patients upper extremities in a neutral, center line position of the patient's body, or moving the upper extremities from possible entanglements.

### SUMMARY OF THE INVENTION

The present invention relates to a non-behavioral medical restraint which keeps a patient in proper alignment in a bed.

The present invention relates to a non-behavioral medical restraint device comprising: a base; four adjustable straps; groin straps; and an abdomen strap.

It is an object of the present invention for the restraint to decrease friction between the patient's skin and bed sheets.

It is an object of the present invention for the restraint to prevent patients from doing harm to themselves.

It is an object of the present invention for the restraint to prevent patients from falling out of bed.

It is an object of the present invention for the restraint to prevent patients from self extubating.

It is an object of the present invention for the restraint to prevent patients from pulling out necessary medical devices.

It is an object of the present invention for the restraint to decrease the risk of aspiration by means of keeping a patient sitting upright.

It is an object of the present invention for the restraint to reduce the back injuries among nurses and other health workers by decreasing the need to pull patients up in bed.

There are many types of restraints which help maintain body alignment, however, the present invention, works by supporting the core of the body and keeping it close to the bed.

It is an object of the present invention for the restraint device to be used with or without other types of restraints such as extremity restraints and vest restraints.

It is an object of the present invention for the restraint device to be made of faux sheepskin. Faux sheepskin helps reduce pressure on skin on boney prominences and helps regulate body temperature.

It is an object of the present invention for the restraint device to be made of a single sheet of faux sheepskin.

It is an object of the present invention for there to be buckles of the top and bottom corners of the restraint device.

It is an object of the present invention for the buckles to be plastic snap buckles.

It is an object of the present invention for the plastic snap buckles to attach to a connecting end piece of the plastic buckle which is looped through the bed frame by a webbing loop.

It is an object of the present invention for the webbing loop to be made of nylon.

It is an object of the present invention that once the buckles are snapped together securely, the base is tightly secured to the bed, as this is the foundation of the device.

It is an object of the present invention for a plastic snap buckle to be located at the side edge of the base midline.

It is an object of the present invention for this buckle to be snapped together with the next strap of the device.

It is an object of the present invention at the midline bottom of the restraint device to have attached an inverted triangle strap which contains no center piece of material.

It is an object of the present invention for the triangle strap to be made of faux sheepskin.

It is an object of the present invention for an approximate 3″ space to be located between the straps at the point of attachment.

It is an object of the present invention for the strap to be made to come between the patient's legs and flush against the patient's inner groin and come up and over the patient's upper abdomen.

It is an object of the present invention for a plastic snap buckle to be located at the ends of the strap.

It is an object of the present invention for the buckles to attach to the two edge midline buckles located on the base.

It is an object of the present invention to have an abdominal strap which is approximately 6″ wide vertically.

It is an object of the present invention for the abdominal strap to be equipped with two buckles at the lateral sides.

It is an object of the present invention that the straps come across the patient's groin, slip through these buckles on the abdominal strap.

It is an object of the present invention for the device to be adjusted according to the patient's size.

### BRIEF DESCRIPTION OF THE DRAWINGS

FIG. 1 is a view of the restraint device placed around a user.

### DETAILED DESCRIPTION OF THE DRAWINGS

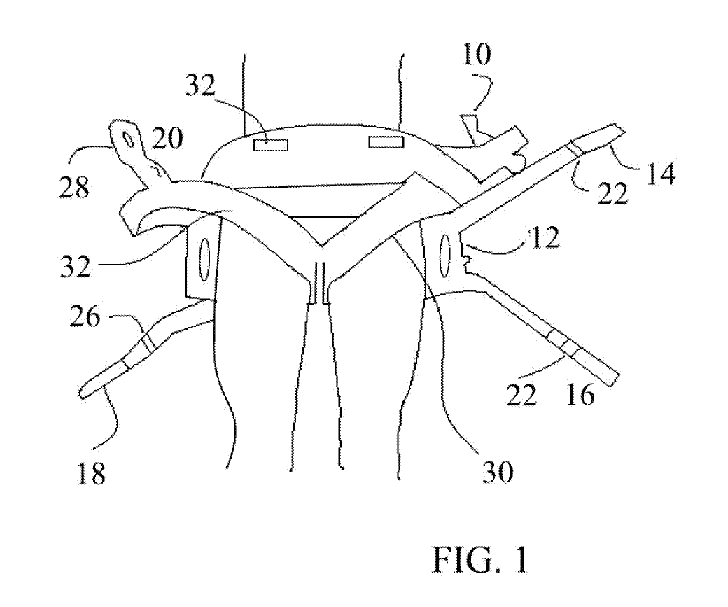

FIG. 1 shows the device 10 having the base 12 which comprises the fauk sheepskin. The device has four adjustable straps 14, 16, 18, 20 having buckles 22, 24, 26 and 28. The device further has groin straps 30 and 32 and a strap abdomen strap 32.

1. A non-behavioral medical restraint device comprising:
a base;
four adjustable straps;
groin straps; and an
abdomen strap.

2. The device of claim 1 wherein said device keeps a patient in proper alignment in a bed.

3. The device of claim 1 wherein said device decreases friction between a patient's skin and bed sheets.

4. The device of claim 1 wherein said device prevents a patient from doing harm to themselves.

5. The device of claim 1 wherein said device prevents a patient from falling out of bed.

6. The device of claim 1 wherein said device prevents a patient from self extubating.

7. The device of claim 1 wherein said device prevents a patient from pulling out necessary medical devices.

8. The device of claim 1 wherein said device decreases the risk of aspiration by keeping a patient sitting upright.

9. The device of claim 1 wherein said device reduces back injuries among nurses and other health workers by decreasing need to pull patients up in bed.

10. The device of claim 1 wherein said device supports core of body and keeps it close to bed.

11. The device of claim 1 wherein said device is used with or without other types of restraints such as extremity restraints and vest restraints.

12. The device of claim 1 wherein said device is made of faux sheepskin.

13. The device of claim 1 wherein said device is made of a single sheet of faux sheepskin.

14. The device of claim 1 wherein said adjustable straps have buckles

15. The device of claim 14 wherein said buckles attach to a connecting end piece of the said buckle which is looped through a bed frame by a webbing loop.

16. The device of claim 15 wherein said webbing loop is made of nylon.

17. The device of claim 14 wherein once said buckles are snapped together securely, said base is tightly secured to bed.

18. The device of claim 1 wherein at midline bottom said device said groin straps and abdomen strap comprises an inverted triangle strap which contains no center piece of material.

19. The device of claim 18 wherein said inverted triangle strap is made to come between patient's legs and flush against patient's inner groin and come up and over patient's upper abdomen.
